(19) United States
(12) Patent Application Publication
(10) Pub. No.: US 2026/0354799 A1
(43) Pub. Date: Nov. 12, 2026

(54) HIGH INTENSITY FOCUSED ULTRASOUND (HIFU) BASED TISSUE ELASTICITY MAPPING FOR TREATMENT PLANNING

(71) Applicant: University of Washington, Seattle, WA (US)

(72) Inventors: Tatiana D. Khokhlova, Seattle, WA (US); Oleg A. Sapozhnikov, Seattle, WA (US); Minho Song, Seattle, WA (US); George R. Schade, Seattle, WA (US); Gerald Lee, Seattle, WA (US)

(73) Assignee: University of Washington, Seattle, WA (US)

(21) Appl. No.: 19/663,425

(22) Filed: Apr. 30, 2026

Related U.S. Application Data
(60) Provisional application No. 63/797,666, filed on Apr. 30, 2025.

(51) Int. Cl. A61B 8/00; A61B 8/08

(52) U.S. Cl. CPC A61B 8/485; A61B 8/085; A61B 8/4477; A61B 8/5207

(57) ABSTRACT

A method for classifying biological tissue using high intensity focused ultrasound (HIFU), the method including: displacing the biological tissue by a burst of ultrasound transmitted by a first ultrasound transducer, wherein the first ultrasound transducer is a HIFU transducer; by displacing the biological tissue, generating a shear wave propagation pattern in the biological tissue, wherein the shear wave propagation pattern includes a longitudinal shear wave pattern and a normal shear wave pattern; tracking the shear wave propagation pattern by a second ultrasound transducer that is an imaging ultrasound transducer; and by tracking the shear wave propagation pattern, mapping a tissue stiffness of the biological tissue. A system including: a first ultrasound transducer that is a high intensity focused (HIFU) transducer and a second ultrasound transducer, wherein the second ultrasound transducer is configured for mapping an elastic modulus of the biological tissue by tracking the shear wave propagation.

### CROSS-REFERENCE(S) TO RELATED APPLICATION(S)

This application claims the benefit of U.S. Provisional Application 63/797,666, filed Apr. 30, 2025, the entire disclosure of which is incorporated herein.

### STATEMENT OF GOVERNMENT LICENSE RIGHTS

This invention was made with government support under Grant No. R01CA258581 awarded by the National Institutes of Health. The government has certain rights in the invention.

### BACKGROUND

Thermal or mechanical tumor ablation with high-intensity focused ultrasound (HIFU) has found widespread use in the past decade, as a non-invasive alternative to surgical excision. In particular, tumor ablation is the most common local treatment procedure in prostate cancer (PCa) ablation, and is typically performed endorectally, under ultrasound (US) imaging guidance.

The US imaging probe is typically integrated coaxially with the therapy transducer for pre-treatment planning and targeting, as well as monitoring the treatment progress. PCa, however, is known to be challenging to localize with US imaging, and even contrast-enhanced magnetic resonance (MR) fusion does not provide an accurate tumor outline. Conversely, US methods that rely on measuring the increased stiffness of PCa tumor—acoustic radiation force imaging (ARFI) and shear wave elastography (SWE) may assist in delineating the tumor accurately. Both methods rely on emitting a longer (up to a few hundred microseconds) burst that, being absorbed by tissue, induces acoustic radiation force (ARF).

The resulting micron-scale tissue displacement in the axial direction launches a shear wave (SW). The velocity of maximum displacement or velocity in the SW, propagating sideways is then measured at a fixed distance (ARFI) or in 2D (SWE) through speckle tracking with short US imaging pulses. Limitations of those methods in the context of HIFU ablation—and PCa ablation in particular—are the reduced push capability at relevant tissue depths and the presence of a water-filled standoff between the probe and the tissue.

Accordingly, there remains a need for improved methods and systems for classifying biological tissue using HIFU.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the present disclosure provides a method for classifying biological tissue using high intensity focused ultrasound (HIFU), the method including: displacing the biological tissue by a burst of ultrasound transmitted by a first ultrasound transducer, wherein the first ultrasound transducer is a high intensity focused ultrasound (HIFU) transducer; by displacing the biological tissue, generating a shear wave propagation pattern in the biological tissue, wherein the shear wave propagation pattern includes a longitudinal shear wave pattern and a normal shear wave pattern; tracking the shear wave propagation pattern by a second ultrasound transducer that is an imaging ultrasound transducer; and by tracking the shear wave propagation pattern, mapping a tissue stiffness of the biological tissue.

In some embodiments, operational parameters of the first ultrasound transducer include a duration of the burst of between about 50 μs and about 500 μs.

In some embodiments, operational parameters of the first ultrasound transducer include a frequency of the first ultrasound transducer that is lower than a frequency of the imaging transducer.

In some embodiments, the second ultrasound transducer is configured for collimated A-line ultrasound imaging.

In some embodiments, a tissue abnormality is present when a speed of the longitudinal shear wave pattern is above a predetermined threshold.

In some embodiments, the tissue abnormality is indicative of a fibrous tissue characterized by a first stiffness that is higher than a second stiffness of surrounding tissue.

In some embodiments, the tissue abnormality is indicative of a tumor tissue characterized by a first stiffness that is higher than a second stiffness of surrounding tissue.

In some embodiments, the tumor is a prostate tumor.

In some embodiments, tracking the shear wave propagation pattern consists of tracking the longitudinal shear wave pattern.

In some embodiments, a tissue abnormality is present when signal attenuation of the longitudinal shear wave pattern is above a predetermined threshold.

In some embodiments, the tissue abnormality is indicative of a tissue having a first viscosity that is higher than a second viscosity of a surrounding tissue.

In some embodiments, the method further includes identifying a location of a tissue abnormality based on an elastic modulus of the biological tissue.

In some embodiments, the location is obtained from a plurality of time-offset A-line images.

In some embodiments, the method further includes identifying a type of the tissue abnormality based on the elastic modulus of the biological tissue.

In some embodiments, the second ultrasound transducer is configured for tracking the shear wave propagation pattern based on a speckle displacement.

In some embodiments, the speckle displacement is indicative of tissue movement.

In some embodiments, the speckle displacement takes place over a time window of about 1 ms to about 50 ms, or of about 1 ms to about 10 ms.

In some embodiments, the HIFU transducer is a phased array HIFU transducer.

In some embodiments, the biological tissue is displaced away from a center axis of the phased array HIFU transducer.

In some embodiments, the imaging transducer is focused away from the center axis of the phased array HIFU transducer.

In some embodiments, the imaging transducer is a single element transducer.

In some embodiments, the imaging transducer is a phased array transducer.

In some embodiments, displacing the biological tissue by a burst of ultrasound transmitted takes place over a first range of scanning angles.

In some embodiments, the first range of scanning angles is between about 5° with respect to the acoustic axis and about 45° with respect to the acoustic axis.

In some embodiments, the HIFU transducer is configured to generate a HIFU beam having a distribution of pressure that includes a plurality of edge lobes.

In some embodiments, the plurality of edge lobes are axially symmetric in at least one dimension.

In an aspect, the present disclosure provides a method for classifying biological tissue using high intensity focused ultrasound (HIFU), the method including: based on a prior imaging, placing an ultrasound probe on patient's body proximate to the biological tissue of interest, the ultrasound probe including a first ultrasound transducer and a second ultrasound transducer; displacing the biological tissue by a plurality of bursts of ultrasound transmitted by the first ultrasound transducer, wherein the first ultrasound transducer is a high intensity focused (HIFU) transducer, wherein individual bursts of the plurality of bursts are characterized by differing angles or focal areas; by displacing the biological tissue by individual bursts of ultrasound, generating corresponding shear wave propagation pattern in the biological tissue, wherein the shear wave propagation pattern includes longitudinal shear wave pattern and a normal shear wave pattern; tracking the shear wave propagation pattern by the second ultrasound transducer that is an imaging ultrasound transducer; and by tracking the shear wave propagation pattern, mapping a tissue stiffness of the biological tissue.

In some embodiments, the biological tissue is a prostate tissue of a patient.

In some embodiments, mapping the tissue stiffness maps: a stiffness value and a location of an area with increased stiffness.

In some embodiments, the prior imaging is a CTE imaging or an MRI imaging.

In an aspect, the present disclosure provides a system for classifying biological tissue using high intensity focused ultrasound (HIFU), the system including: a first ultrasound transducer that is a high intensity focused (HIFU) transducer configured for: displacing the biological tissue by transmitting a burst of ultrasound, and by displacing the biological tissue, generating a shear wave propagation pattern in the biological tissue, wherein the shear wave propagation pattern includes longitudinal shear wave pattern and a normal shear wave pattern; and a second ultrasound transducer that is an imaging ultrasound transducer, wherein the second ultrasound transducer is configured for mapping an elastic modulus of the biological tissue by tracking the shear wave propagation.

In some embodiments, the system further includes: a controller configured for identifying a location of abnormal tissue based on the elastic modulus of the biological tissue.

In some embodiments, the controller is further configured for: identifying a size and a shape of the abnormal tissue based on the elastic modulus of the biological tissue.

In some embodiments, the controller is further configured for: identifying a type of abnormal tissue based on the elastic modulus of the biological tissue.

In some embodiments, the second ultrasound transducer is configured for tracking the shear wave propagation pattern based on speckle displacement.

In some embodiments, the speckle displacement takes place over a time window of about 1 ms to about 50 ms, or about 1 ms to about 10 ms.

In some embodiments, the HIFU transducer is a phased array HIFU transducer.

In some embodiments, the phased array HIFU transducer is configured for displacing the biological tissue away from a center axis of the phased array HIFU transducer.

In some embodiments, the imaging ultrasound transducer is focused away from the center axis of the phased array HIFU transducer.

In some embodiments, the imaging ultrasound transducer is a single element ultrasound transducer.

In some embodiments, the HIFU transducer is configured to generate a HIFU beam having a distribution of pressure that includes a plurality of edge lobes.

In some embodiments, the plurality of edge lobes are axially symmetric in at least one dimension.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 depicts a schematic illustration of an ultrasound probe interrogating a tissue in a first configuration, according to embodiments of the present disclosure;

FIG. 2 depicts a schematic illustration of an ultrasound probe interrogating a tissue in a second configuration, according to embodiments of the present disclosure;

FIG. 3 depicts a flow chart of a process according to embodiments of the present disclosure;

FIG. 4 depicts a flow chart of a process according to embodiments of the present disclosure;

FIG. 5A depicts an experimental setup for high-intensity focused ultrasound (HIFU)-based longitudinal shear wave elasticity (LSWE) measurements, according to embodiments of the present disclosure;

FIG. 5B depicts normalized HIFU intensity distribution, according to embodiments of the present disclosure;

FIG. 5C depicts a pulsing sequence utilized for LSWE measurement, according to embodiments of the present disclosure;

FIG. 5D depicts SWE images of the PVA tissue mimicking phantoms with different elastic moduli obtained with a clinical system, according to embodiments of the present disclosure;

FIG. 6A depicts numerically simulated axial displacement fields in the phantom resulting from a 500 μs push from the HIFU transducer at a 0.5 ms timepoint, according to embodiments of the present disclosure;

FIG. 6B depicts numerically simulated axial displacement fields in the phantom resulting from a 500 μs push from the HIFU transducer at a 1.3 ms timepoint, according to embodiments of the present disclosure;

FIG. 6C depicts numerically simulated axial displacement fields in the phantom resulting from a 500 μs push from the HIFU transducer at a 2.2 ms timepoint, according to embodiments of the present disclosure;

FIG. 6D depicts waveforms of displacement for SW (low values) and LSW (high values) at different points on transverse and longitudinal axes, according to embodiments of the present disclosure;

FIG. 6E depicts normalized distribution of displacement or velocity along lateral coordinates over SW tracking time, according to embodiments of the present disclosure;

FIG. 6F depicts normalized distribution of displacement or velocity along axial coordinates over SW tracking time, according to embodiments of the present disclosure;

FIG. 7A depicts numerically simulated vibrational velocity fields in the phantom resulting from a 500 μs push from the HIFU transducer at a 0.5 ms timepoint, according to embodiments of the present disclosure;

FIG. 7B depicts numerically simulated vibrational velocity fields in the phantom resulting from a 500 μs push from the HIFU transducer at a 1.3 ms timepoint, according to embodiments of the present disclosure;

FIG. 7C depicts numerically simulated vibrational velocity fields in the phantom resulting from a 500 μs push from the HIFU transducer at a 2.2 ms timepoint, according to embodiments of the present disclosure;

FIG. 7D depicts waveforms of vibrational velocity for SW (low values) and LSW (high values) at different points on transverse and longitudinal axes, according to embodiments of the present disclosure;

FIG. 7E depicts normalized distribution of displacement or velocity along lateral coordinates over SW tracking time, according to embodiments of the present disclosure;

FIG. 7F depicts normalized distribution of displacement or velocity along axial coordinates over SW tracking time, according to embodiments of the present disclosure;

FIG. 8A depicts a Gaussian-shaped beam at a 0.5 ms timepoint, according to embodiments of the present disclosure;

FIG. 8B depicts a Gaussian-shaped beam at a 1.3 ms timepoint, according to embodiments of the present disclosure;

FIG. 8C depicts a Gaussian-shaped beam at a 2.2 ms timepoint, according to embodiments of the present disclosure;

FIG. 8D depicts waveforms of displacement for SW (low values) and LSW (high values) at different points on transverse and longitudinal axes, according to embodiments of the present disclosure;

FIG. 8E depicts normalized distribution of displacement or velocity along lateral coordinates over SW tracking time, according to embodiments of the present disclosure;

FIG. 8F depicts normalized distribution of displacement or velocity along axial coordinates over SW tracking time, according to embodiments of the present disclosure;

FIG. 9A depicts a measurement of the longitudinal shear wave speed c_{LSW} in homogenous phantoms of different stiffness, according to embodiments of the present disclosure;

FIG. 9B depicts a measurement of the longitudinal shear wave speed c_{LSW} in homogenous phantoms of different stiffness, according to embodiments of the present disclosure;

FIG. 9C depicts a measurement of the longitudinal shear wave speed c_{LSW} in homogenous phantoms of different stiffness, according to embodiments of the present disclosure;

FIG. 9D depicts the arrival time of maximum negative vibrational velocity used to obtain c_{LSW} in the soft phantom, according to embodiments of the present disclosure;

FIG. 9E depicts an average Young's moduli of the three phantoms measured by the clinical SWE system (narrow hatching, “classic SWE”) and calculated from c_{LSW} of the waves propagating towards (wide hatching) and away (white) from the transducer, according to embodiments of the present disclosure;

FIG. 10A depicts direct plane wave imaging of shear wave propagation following a HIFU push in the softest phantom at 1.0 ms, according to embodiments of the present disclosure;

FIG. 10B depicts direct plane wave imaging of shear wave propagation following a HIFU push in the softest phantom at 1.5 ms, according to embodiments of the present disclosure; and

FIG. 10C depicts direct plane wave imaging of shear wave propagation following a HIFU push in the softest phantom at 2.6 ms, according to embodiments of the present disclosure.

### DETAILED DESCRIPTION

The present disclosure provides, in some embodiments, methods and systems for classifying biological tissue using high intensity focused ultrasound (HIFU).

In this regard, in some embodiments the present disclosure relates generally to a HIFU-push method of shear wave elastography imaging (SWEI) that would overcome the limitations in the context of prostate cancer (PCa) ablation planning. In some embodiments, 1.5 MHz HIFU transducer integrated coaxially with an ultrasound (US) imaging probe produced a 500-microsecond push at its focus through acoustic radiation force in homogenous tissue-mimicking phantoms with different stiffness representative of benign and cancerous prostatic tissue. Such integration of the HIFU transducer with the US imaging probe alleviates some of the shortcomings of the prior art probes, which lack push-capable US imaging probes integrated with HIFU transducers. The US imaging probe was used to track the resulting speckle displacement along the HIFU axis over a 50 ms time window.

In some embodiments, the Young's moduli of the phantoms were calibrated by a clinical SWEI system (SSI Aixplorer) and were 7.5, 30, and 59 kPa. Finite differences numerical modelling of SW generation and propagation in the phantoms revealed both side-propagating shear wave (SW), and those propagating along the HIFU axis, towards and away from the transducer—“longitudinal” SW (LSW). These LSW formed due to constructive interference of the SW generated throughout the conical HIFU beam, and their propagation speed was higher than SW velocity by a constant factor dependent on the HIFU field shape. Measurement of LSW propagation speed may map Young's modulus distribution along the HIFU axis, with average values in close agreement with the calibration for the three phantoms.

### Systems

In an aspect, the present disclosure provides a system for classifying biological tissue using high intensity focused ultrasound (HIFU), the system including: a first ultrasound transducer that is a high intensity focused (HIFU) transducer configured for: displacing the biological tissue by transmitting a burst of ultrasound, and by displacing the biological tissue, generating a shear wave propagation pattern in the biological tissue, wherein the shear wave propagation pattern includes longitudinal shear wave pattern and a normal shear wave pattern; and a second ultrasound transducer that is an imaging ultrasound transducer, wherein the second ultrasound transducer is configured for mapping an elastic modulus of the biological tissue by tracking the shear wave propagation.

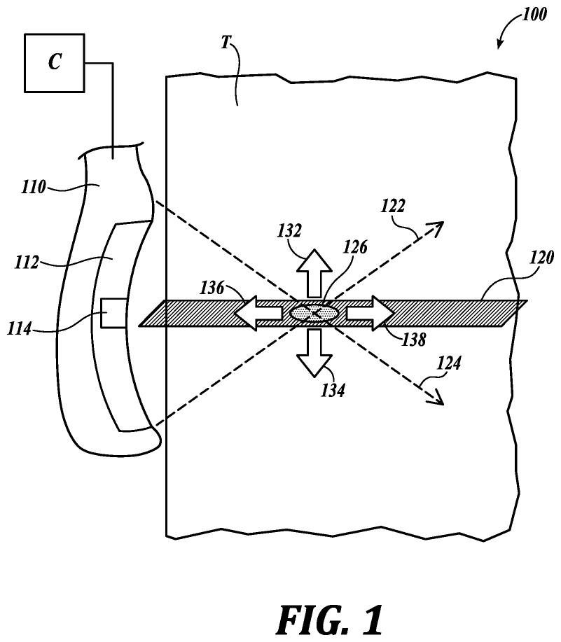

In this regard, FIG. 1 depicts schematically a system 100 for classifying biological tissue using HIFU when system 100 is positioned proximate a tissue T of a patient. System 100 includes a probe 110 which includes a first ultrasound transducer that is a HIFU transducer array 112 and a second ultrasound transducer that is an imaging ultrasound transducer array 114. In some embodiments, the HIFU transducer 112 and/or imaging ultrasound transducer 114 may be single monolithic ultrasound transducer(s). A controller C is depicted as being operatively coupled to the probe 110, though it should be understood that while in some embodiments the probe 110 is coupled to a controller C, controller C itself may be an optional part of the system 100. In operation, the controller C may provide instructions for the HIFU transducer 112 and imaging transducer 114 (e.g., frequency, amplitude, phase delays affecting the angle of the ultrasound beam, etc.), or may process the ultrasound images to determine relevant parameters. In different embodiments, the controller C may be a digital controller (e.g., laptop computer, desktop computer, smart phone, tablet, etc.) or an analog controller (e.g., an op-amp with supporting electronics, etc.).

When system 100 is in operation, a tracking plane 120 may be defined based on the relative position of imaging ultrasound transducer array 114 to the tissue T of the patient. The HIFU transducer array 112 may be configured to displace the tissue T by transmitting a burst of ultrasound, and by displacing the tissue T, generating a shear wave propagation pattern in the tissue. As mentioned above, the HIFU transducer array 112 may, in some embodiments, be a single transducer. In some embodiments, the HIFU transducer array 112 may be a phased array ultrasound transducer. In this regard, FIG. 1 depicts a first edge lobe 122 and a second edge lobe 124 which combine to generate a push region (also referred to as “a target region” or “a push” for simplicity and brevity) 126 in the tissue T. The shear wave propagation pattern generated at the push region 126 is depicted to include a first normal shear wave 132, a second normal shear wave 134, a first longitudinal shear wave 136, and a second longitudinal shear wave 138.

As the first normal shear wave 132, second normal shear wave 134, first longitudinal shear wave 136, and second longitudinal shear wave 138 propagate through the tissue T, the imaging ultrasound transducer array 114 tracks the shear wave propagation pattern within tracking plane 120, and by so tracking determines the elastic modulus of the tissue T in a region of the patient.

Controller C is depicted as being operatively coupled to the probe 110, the controller including at least one processor and a computer-readable medium having computer-executable instructions stored thereon that, in response to execution by the at least one processor, cause the controller to perform operations including displacing the biological tissue by a burst of ultrasound transmitted by a first ultrasound transducer, wherein the first ultrasound transducer is a high intensity focused ultrasound (HIFU) transducer; by displacing the biological tissue, generating a shear wave propagation pattern in the biological tissue, wherein the shear wave propagation pattern includes a longitudinal shear wave pattern and a normal shear wave pattern; tracking the shear wave propagation pattern by a second ultrasound transducer that is an imaging ultrasound transducer; and by tracking the shear wave propagation pattern, mapping a tissue stiffness of the biological tissue. In this regard, when controller C is operatively coupled to probe 110, the system may be configured to perform any of the methods described herein.

FIG. 1 thus depicts an example system 100 in use with a patient where the push region 126 occurs within the tracking plane 120. However, it should be understood that the systems according to the present disclosure may also determine the elastic modulus by generating a push region (target region) in the tissue T in a region separate from the tracking plane 120.

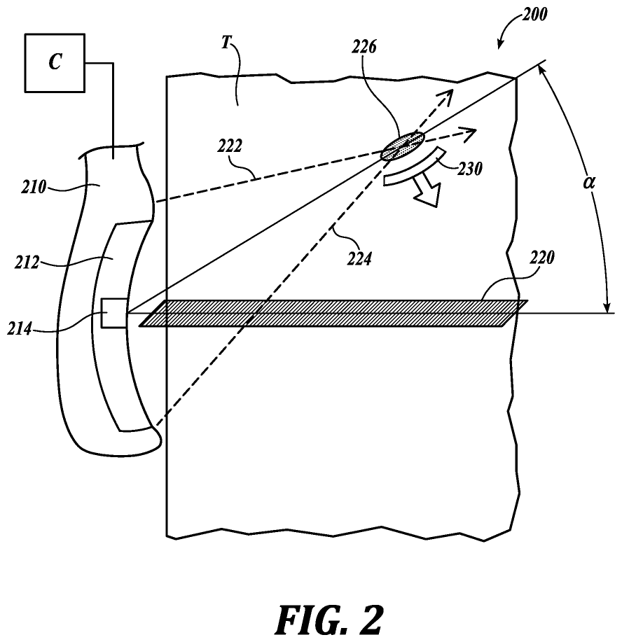

In this regard, FIG. 2 depicts schematically a system 200 for classifying biological tissue using HIFU in position next to a tissue T of a patient. System 200 may, in some embodiments, be an example of system 100. Thus, the components and operation of system 200 will be described analogously to that of system 100, but in the 2xx series.

System 200 includes a probe 210 which includes a first ultrasound transducer that is a HIFU transducer array 212 and a second ultrasound transducer that is an imaging ultrasound transducer array 214. A controller C is depicted as being operatively coupled to the probe 210, though it should be understood that while in some embodiments the probe 210 is coupled to a controller C, controller C itself may be an optional part of the system 200.

When system 200 is in operation, a tracking plane 220 is defined based on the relative position of imaging ultrasound transducer array 214 to the tissue T of the patient. The HIFU transducer array 212 may be configured to displace the tissue T by transmitting a burst of ultrasound, and by displacing the tissue T, generating a shear wave propagation pattern in the tissue. The HIFU transducer array 212 may, in some embodiments, be a single monolithic transducer. In some embodiments, the HIFU transducer array 212 may be a phased array ultrasound transducer. In this regard, FIG. 2 depicts a first edge lobe 222 and a second edge lobe 224 which overlap to generate a push region (target region) 226 in the tissue T. Unlike in FIG. 1, where the push region 126 was within the tracking plane 120, the push region 226 is at an angle α relative to the tracking plane 220. Operation of the ultrasound at the push region 226 thereby generates a shear wave 230.

As the shear wave 230 propagates through the tissue T, the imaging ultrasound transducer array 214 tracks the shear wave 230 propagation pattern as it moves through tracking plane 220, and by so tracking determining the elastic modulus of the tissue T in a region of the patient.

Thus, FIG. 1 and FIG. 2 together demonstrate different mechanisms by which the elastic modulus of tissue T in a patient may be monitored by generating push regions using HIFU at a variety of positions. Specific examples of these various configurations are described further in the Examples.

In some embodiments, the system further includes: a controller configured for identifying a location of abnormal tissue based on the elastic modulus of the biological tissue. In some embodiments, the controller is operatively coupled to the first ultrasound transducer and the second ultrasound transducer, the controller including at least one processor and a computer-readable medium having computer-executable instructions stored thereon that, in response to execution by the at least one processor, cause the controller to perform operations including any of the method steps described herein.

In some embodiments, the controller is further configured for: identifying a size and a shape of the abnormal tissue based on the elastic modulus of the biological tissue.

In some embodiments, the controller is further configured for: identifying a type of abnormal tissue based on the elastic modulus of the biological tissue.

In some embodiments, the second ultrasound transducer is configured for tracking the shear wave propagation pattern based on speckle displacement.

In some embodiments, the speckle displacement takes place over a time window of about 1 ms to about 50 ms, or about 1 ms to about 10 ms.

In some embodiments, the HIFU transducer is a phased array HIFU transducer.

In some embodiments, the phased array HIFU transducer is configured for displacing the biological tissue away from a center axis of the phased array HIFU transducer.

In some embodiments, the imaging ultrasound transducer is focused away from the center axis of the phased array HIFU transducer.

In some embodiments, the imaging ultrasound transducer is a single element ultrasound transducer.

In some embodiments, the HIFU transducer is configured to generate a HIFU beam having a distribution of pressure that includes a plurality of edge lobes.

In some embodiments, the plurality of edge lobes are axially symmetric in at least one dimension.

### Methods

In an aspect, the present disclosure provides a method for classifying biological tissue using high intensity focused ultrasound (HIFU), the method including: displacing the biological tissue by a burst of ultrasound transmitted by a first ultrasound transducer, wherein the first ultrasound transducer is a high intensity focused ultrasound (HIFU) transducer; by displacing the biological tissue, generating a shear wave propagation pattern in the biological tissue, wherein the shear wave propagation pattern includes a longitudinal shear wave pattern and a normal shear wave pattern; tracking the shear wave propagation pattern by a second ultrasound transducer that is an imaging ultrasound transducer; and by tracking the shear wave propagation pattern, mapping a tissue stiffness of the biological tissue.

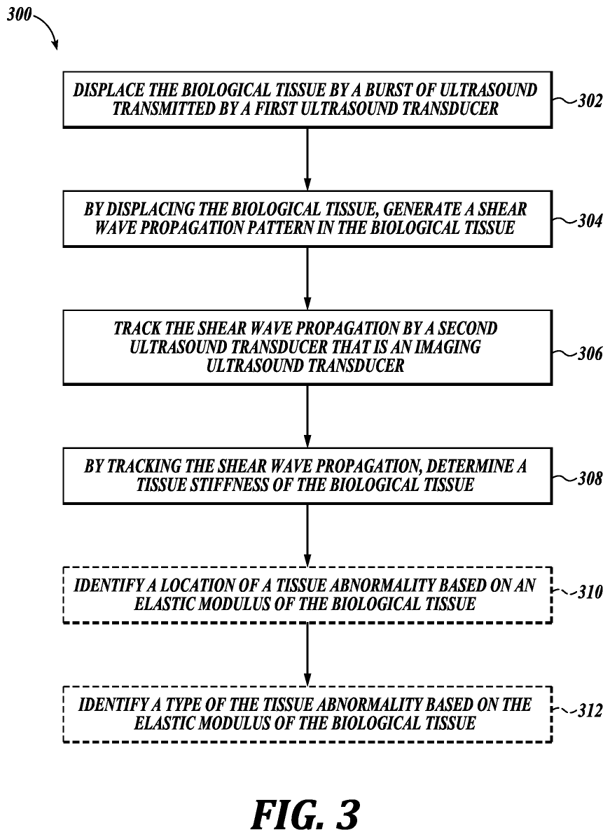

In this regard, FIG. 3 depicts a flow diagram of a method 300 according to the present disclosure. For clarity, method 300 will be described in relation to system 100 as described above with respect to FIG. 1 and/or with respect to system 200 as described above with respect to FIG. 2. However, it should be understood that the methods described herein are not limited to the structures described in system 100, and that such description is intended to be illustrative rather than limiting.

The method 300 begins at process block 302 by displacing the biological tissue T by a burst of ultrasound transmitted by a first ultrasound transducer (HIFU array 112). In some embodiments, this burst of ultrasound may result in a push region such as push region 126 at a location within tracking plane 120. It should be understood, however, that the methods described herein are not so limited, and that the burst of ultrasound may instead result in a push region 126 at a location outside of tracking plane 220, as described above.

Next, at process block 304, the method includes displacing the biological tissue T, thereby generating a shear wave propagation pattern (i.e., first normal shear wave 132, second normal shear wave 134, first longitudinal shear wave 136, second longitudinal shear wave 138, or shear wave 230).

At process block 306, the method includes tracking the shear wave propagation by a second ultrasound transducer that is an imaging ultrasound transducer (i.e., imaging array 114).

Then, at process block 308, the method includes tracking the shear wave propagation, and thereby determining a tissue stiffness of the biological tissue T.

At optional process block 310, the method includes identifying a location of a tissue abnormality based on an elastic modulus (such as may be determined by the tissue stiffness) of the biological tissue T.

Finally, at optional process block 312, the method includes identifying a type of the tissue abnormality (i.e., a fibrous tissue, a tumor tissue, etc.) based on the elastic modulus of the biological tissue T.

In another aspect, the present disclosure provides a method for classifying biological tissue using high intensity focused ultrasound (HIFU), the method including: based on a prior imaging, placing an ultrasound probe on patient's body proximate to the biological tissue of interest, the ultrasound probe including a first ultrasound transducer and a second ultrasound transducer; displacing the biological tissue by a plurality of bursts of ultrasound transmitted by the first ultrasound transducer, wherein the first ultrasound transducer is a high intensity focused (HIFU) transducer, wherein individual bursts of the plurality of bursts are characterized by differing angles or focal areas; by displacing the biological tissue by individual bursts of ultrasound, generating corresponding shear wave propagation pattern in the biological tissue, wherein the shear wave propagation pattern includes longitudinal shear wave pattern and a normal shear wave pattern; tracking the shear wave propagation pattern by the second ultrasound transducer that is an imaging ultrasound transducer; and by tracking the shear wave propagation pattern, mapping a tissue stiffness of the biological tissue.

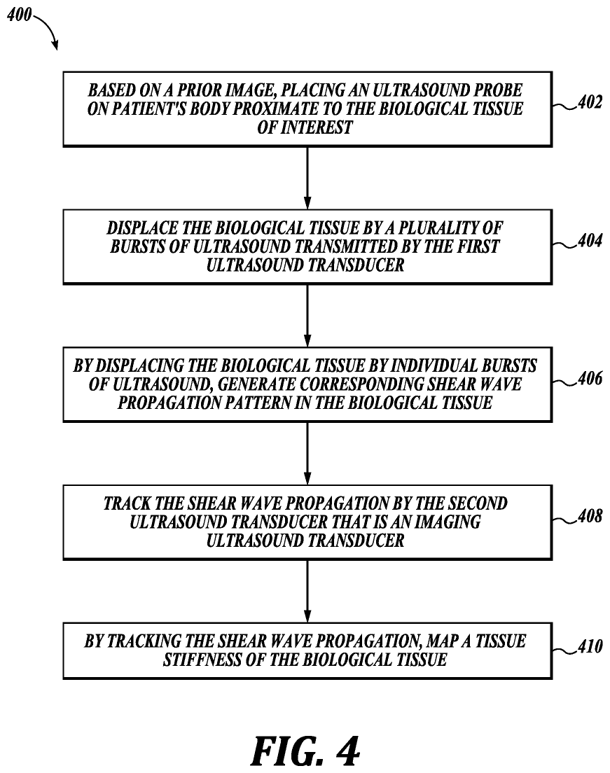

In this regard, FIG. 4 depicts a flow diagram of a method 400 according to the present disclosure. For clarity, method 400 will be described in relation to system 100 as described above with respect to FIG. 1 and/or with respect to system 200 as described above with respect to FIG. 2. However, it should be understood that the methods described herein are not limited to the structures described in system 100, and that such description is intended to be illustrative rather than limiting.

The method 400 begins at process block 402 by, based on a prior image, placing an ultrasound probe (i.e., probe 110) on a patient's body proximate to the biological tissue T of interest.

Next, at process block 404, the method includes displacing the biological tissue T by a plurality of bursts of ultrasound (such as with push region 126 or push region 226) transmitted by the first ultrasound transducer (i.e., HIFU array 112).

At process block 406, the method includes, by displacing the biological tissue T by these individual bursts of ultrasound, generating corresponding shear wave propagation patterns (i.e., first normal shear wave 132, second normal shear wave 134, first longitudinal shear wave 136, second longitudinal shear wave 138, or shear wave 230).

Then, at process block 408, the method includes tracking the shear wave propagation by the second ultrasound transducer that is an imaging transducer (i.e., imaging array 114).

Finally, at process block 410, the method includes, by tracking the shear wave propagation, mapping a tissue stiffness of the biological tissue T.

While process 400 is described with respect to the above method steps, it should be further understood that additional process steps may be used in conjunction with process 400, such as any of the process steps described with respect to process 300, or other process steps described herein.

In some embodiments, operational parameters of the first ultrasound transducer include a duration of the burst of between about 50 μs and about 500 μs.

In some embodiments, operational parameters of the first ultrasound transducer include a frequency of the first ultrasound transducer that is lower than a frequency of the imaging transducer.

In some embodiments, the second ultrasound transducer is configured for collimated A-line ultrasound imaging.

In some embodiments, a tissue abnormality is present when a speed of the longitudinal shear wave pattern is above a predetermined threshold.

In some embodiments, the tissue abnormality is indicative of a fibrous tissue characterized by a first stiffness that is higher than a second stiffness of surrounding tissue.

In some embodiments, the tissue abnormality is indicative of a tumor tissue characterized by a first stiffness that is higher than a second stiffness of surrounding tissue.

In some embodiments, the tumor is a prostate tumor.

In some embodiments, tracking the shear wave propagation pattern consists of tracking the longitudinal shear wave pattern. In this regard, in some embodiments, tracking the shear wave propagation pattern does not include tracking the normal shear wave pattern.

In some embodiments, a tissue abnormality is present when signal attenuation of the longitudinal shear wave pattern is above a predetermined threshold.

In some embodiments, the tissue abnormality is indicative of a tissue having a first viscosity that is higher than a second viscosity of a surrounding tissue.

In some embodiments, the method further includes identifying a location of a tissue abnormality based on an elastic modulus of the biological tissue.

In some embodiments, the location is obtained from a plurality of time-offset A-line images.

In some embodiments, the method further includes identifying a type of the tissue abnormality based on the elastic modulus of the biological tissue.

In some embodiments, the second ultrasound transducer is configured for tracking the shear wave propagation pattern based on a speckle displacement.

In some embodiments, the speckle displacement is indicative of tissue movement.

In some embodiments, the speckle displacement takes place over a time window of about 1 ms to about 50 ms, or of about 1 ms to about 10 ms.

In some embodiments, the HIFU transducer is a phased array HIFU transducer.

In some embodiments, the biological tissue is displaced away from a center axis of the phased array HIFU transducer.

In some embodiments, the imaging transducer is focused away from the center axis of the phased array HIFU transducer.

In some embodiments, the imaging transducer is a single element transducer.

In some embodiments, the imaging transducer is a phased array transducer.

In some embodiments, displacing the biological tissue by a burst of ultrasound transmitted takes place over a first range of scanning angles.

In some embodiments, the first range of scanning angles is between about 5° with respect to the acoustic axis and about 45° with respect to the acoustic axis.

In some embodiments, the HIFU transducer is configured to generate a HIFU beam having a distribution of pressure that includes a plurality of edge lobes.

In some embodiments, the plurality of edge lobes are axially symmetric in at least one dimension.

In some embodiments, the biological tissue is a prostate tissue of a patient.

In some embodiments, mapping the tissue stiffness maps: a stiffness value and a location of an area with increased stiffness.

In some embodiments, the prior imaging is a CTE imaging or an MRI imaging.

### DEFINITIONS

As used herein, a “predetermined threshold” refers to a setpoint above which a tissue abnormality is identified to be present based on the particular measurement parameters (i.e., the speed of the longitudinal shear wave pattern, the attenuation of signal from the longitudinal shear wave pattern) and the subject (i.e., prostate, liver). In this regard, in some embodiments, the predetermined threshold may be determined by the noise in the measurement parameter such that the difference in signal exceeds the noise threshold. In some embodiments, the predetermined threshold may be determined empirically based on the context of the measurement, including consideration of the different types of tissue being measured (liver, prostate, or other), the particular condition being probed for (cancer or fibrosis), and the like. It should be understood that such a predetermined parameter may thus, in some embodiments, be determined by a user (such as a clinician) of the methods and systems described herein based on empirical data in a given clinical context, and that determination of an appropriate threshold for a given measurement is within the abilities of one of ordinary skill in the art.

### EXAMPLES

The following examples are provided to illustrate various embodiments of the present disclosure. In this regard, while reference to FIGS. 1-4 may be made for clarity, it should be understood that each of the examples below may include additional embodiments beyond those that are depicted in FIGS. 1-4.

### Example 1

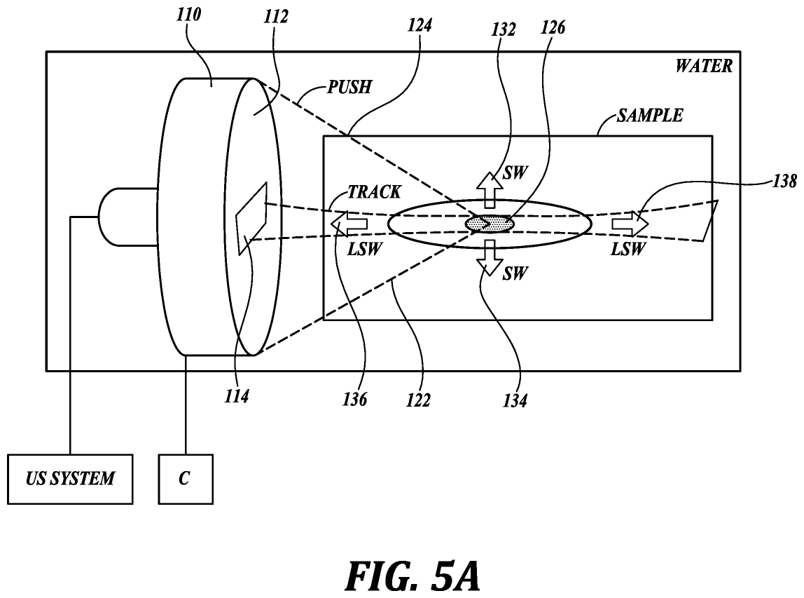

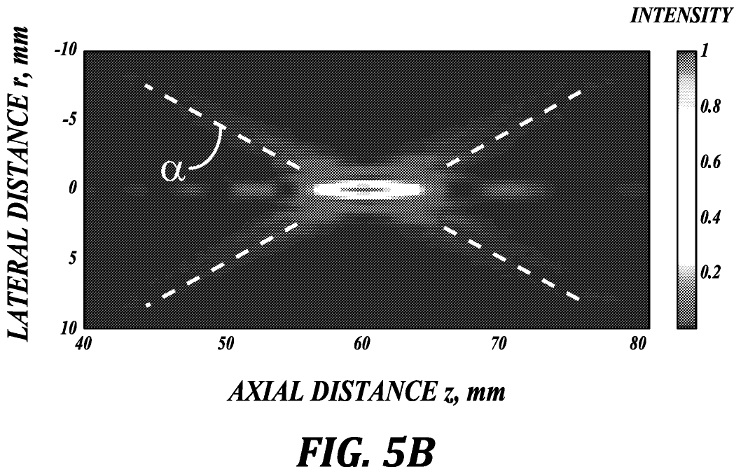

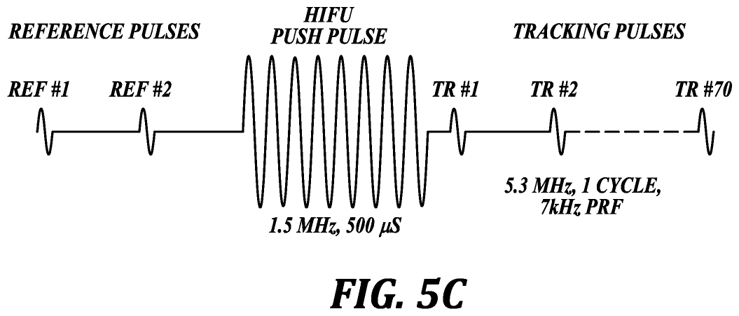

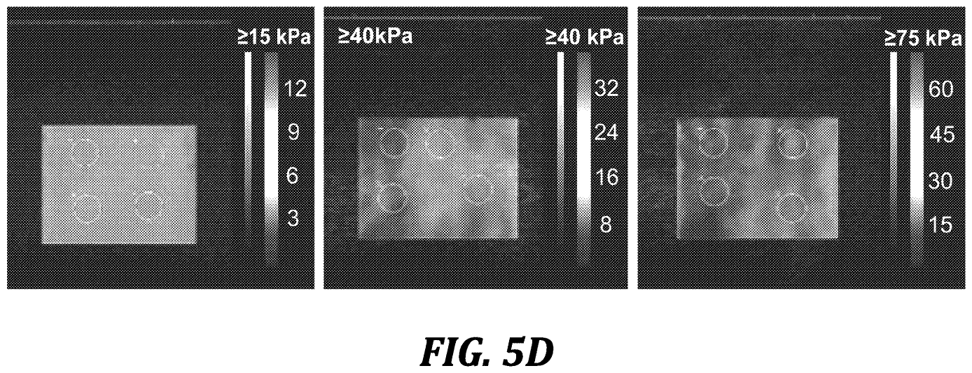

FIG. 5A depicts an experimental setup for HIFU-based longitudinal shear wave (LSWE) elasticity measurements.

FIG. 5B depicts normalized HIFU intensity distribution within the axial plane used in numerical simulations, where the prefocal and postfocal edge lobes are at an angle α=28° with acoustic axis that are common for spherically focused HIFU sources.

FIG. 5C depicts a pulsing sequence utilized for LSWE measurement.

FIG. 5D depicts SWE images of the PVA tissue mimicking phantoms with different elastic moduli obtained with a clinical system.

The present disclosure describes an elastography method that circumvents limitations of using a HIFU-induced push region and US imaging-based tracking of the resulting SW propagation (FIG. 5C). The method was investigated both numerically and experimentally in homogenous tissue-mimicking phantoms with calibrated elastic moduli within the range typical for benign prostate tissues and PCa.

Numerical simulation of the shear wave propagation resulting from a HIFU push was performed with finite differences, assuming axial symmetry of the HIFU field. The near-field interaction between longitudinal and shear waves previously described for elastic solids was not considered in the simulation due to the effect being negligible outside the HIFU push region, where displacement measurements were performed. In the subsequent notation the z-axis is the HIFU beam axis of symmetry, r is the transverse coordinate (distance from the axis), and u_{z} is the axial component of the medium displacement during the propagation of the SW generated by acoustic radiation force. Note that only the axial displacement component u_{z}, and not the radial component uᵣ, was modeled because it corresponds to the displacement measured experimentally. The wave equation for SW propagating with a velocity c_{SW}=\sqrt{μ/ρ}, where μ is the shear modulus of the medium, ρ is the density, with a source term described as a force F_{z} acting on a unit volume is:

$$\frac{{\partial }^{2} {u}_{z}}{\partial {t}^{2}} - {c}_{SW}^{2} Δ {u}_{z} = \frac{{F}_{z}}{ρ} \qquad ( 1 )$$

The acoustic radiation force F_{z} arises from absorption of the ultrasound push region burst in the medium and can be expressed through the axial component of the wave intensity I_{z} as follows: F_{z}=2αI_{z}/c₀. Here α is the ultrasound attenuation coefficient, c₀ is the propagation velocity of longitudinal waves (the speed of sound). For numerical modeling, it is convenient to rewrite this equation in terms of velocity v_{z}=∂u_{z}/∂t and auxiliary variables S_{zz} and S_{zr}:

$$\frac{\partial {v}_{z}}{\partial t} = {C}_{SW}^{2} [ \frac{1}{r} \frac{\partial ( {rS}_{zr} )}{\partial r} + \frac{\partial {S}_{zz}}{\partial z} ] + \frac{2 α {I}_{z}}{ρ {c}_{0}} , \qquad ( 2 )$$

$$\frac{\partial {S}_{zz}}{\partial t} = \frac{\partial {v}_{z}}{\partial z} , \qquad ( 3 )$$

$$\frac{\partial {S}_{zr}}{\partial t} = \frac{\partial {v}_{z}}{\partial r} . \qquad ( 4 )$$

The axial component of intensity I_{z} for a harmonic longitudinal ultrasound wave is expressed in terms of the complex amplitudes of the acoustic pressure P and the axial component of the vibrational velocity V_{z} (not to be confused with the velocity component v_{z} in a shear wave mentioned above): I_{z}=Re(V_{z}P*)/2, where the asterisk denotes the complex conjugate. The values P* and V_{z} were calculated based on the Rayleigh integral. A 1.5 MHz frequency transducer in the form of a uniformly vibrating spherical bowl with geometric dimensions corresponding to the nominal size of the source used in the experiment was used as a model source: focal distance 60 mm, aperture diameter 73 mm, and the central opening diameter 30 mm. The distribution of I_{z} within the axial plane of the HIFU transducer is presented in FIG. 5B. Note the conically-shaped low amplitude prefocal and postfocal HIFU beam at an angle α=28° to the HIFU axis. To solve Eqs. (2)-(4) in finite differences, discretization was performed using a central difference scheme with staggered grids in both space and time. The velocity and strain tensor of the medium were initially set to zero.

The experimental setup illustrated in FIG. 5A included a spherically focused 1.5 MHz HIFU transducer (aperture 73 mm, F-number 0.83, focal area dimensions 1.8×12 mm at the null level) coaxially integrated with an US imaging probe (ATL P7-4 phased array). The HIFU transducer was driven by a custom-built class D amplifier, and the imaging probe was operated by Verasonics V1 system (Verasonics Inc., Kirkland, WA, USA). The HIFU transducer focus was placed at the depth of 12 mm within a tissue-mimicking phantom (64 mm from the transducer; 52 mm water standoff), and a push was produced with a 500-microsecond burst, peak positive pressure of 21.9 MPa, and peak negative pressure of 9.7 MPa.

Under these settings, cavitation was not observed in any of the experiments. The US imaging probe emitted 5.3 MHz two reference tracking pulses before the push, and 70 tracking pulses after the push at pulse repetition frequency (PRF) of 7 kHz, beamformed along the HIFU axis, with a maximum depth of 7 cm (FIG. 5C). The received echoes were sampled at 45 MHz and then stored for offline processing. In a separate set of experiments aimed at observing the full SW field, the tracking pulses were not beamformed and instead were emitted as plane waves.

To obtain the distribution of SW-induced displacement along the HIFU axis, the radio-frequency (RF) signals from each tracking pulse were performed with dynamic receive beamforming along the HIFU axis, upsampled 30-fold to 1.35 GHz and correlated via normalized cross-correlation (NCC) to the reference signal with a 0.58 mm (2λ) kernel. The displacement values associated with NCC amplitude below 0.975 were discarded. In the case of 2D displacement reconstruction the same processing as above was used for 53 tracking lines—−4.5 mm to the left and 4.5 mm to the right of the HIFU axis—spaced by 0.17 mm (pitch size of the imaging probe), except the threshold for acceptable NCC amplitude was set lower, at 0.9.

The tissue-mimicking phantoms were fabricated by pouring 6% polyvinyl alcohol (PVA) with 3% of aluminum powder into cuboid molds of 5×5×5 cm size and freezing the mixture for 24 hours. The different elastic moduli were achieved by varying the number of freeze-thaw cycles from 1 to 3, with the larger number of freeze-thaw cycles producing stiffer phantoms. The ultrasound attenuation coefficient and the speed of sound in the phantoms were characterized with substitution method, as previously described, and were 0.050±0.001 Np/cm and 1486±7.3 m/s, respectively, across the three phantoms. The spatial homogeneity of the phantoms elasticity was confirmed by imaging them with a clinical SWE system (Aixplorer, Supersonic Imagine, Aix-en-Provence, France) with an L14-5 imaging probe (FIG. 5D). The Young's moduli within the phantoms ranged within 7.1-8.0 kPa, 26.9-32.7 kPa, and 55.6-62.4 kPa for the softest to the stiffest phantom, respectively.

### Example 2

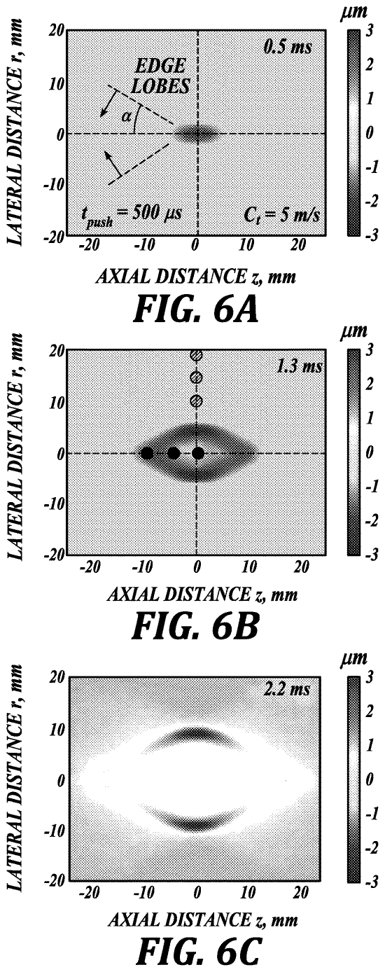

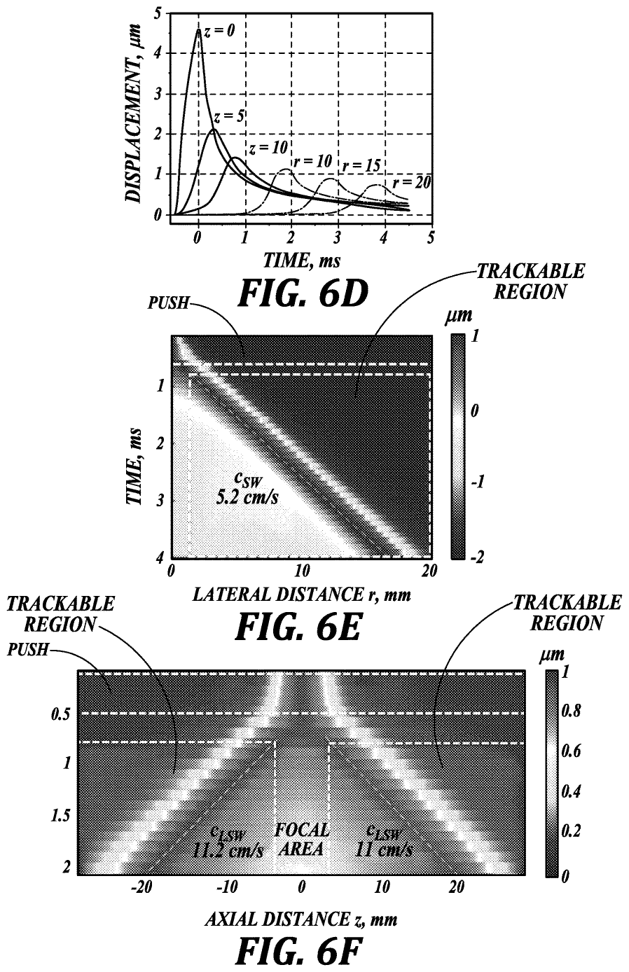

FIG. 6A depicts numerically simulated axial displacement fields in the phantom resulting from a 500 μs push from the HIFU transducer at a 0.5 ms timepoint (start of the push corresponds to zero time point). HIFU is incident from the left of the images. Hashed dots on the vertical and solid dots on the horizontal axes denote the locations of SWE waveform detection.

FIG. 6B depicts numerically simulated axial displacement fields in the phantom resulting from a 500 μs push from the HIFU transducer at a 1.3 ms timepoint (start of the push corresponds to zero time point). HIFU is incident from the left of the images. Hashed dots on the vertical and solid dots on the horizontal axes denote the locations of SWE waveform detection.

FIG. 6C depicts numerically simulated axial displacement fields in the phantom resulting from a 500 μs push from the HIFU transducer at a 2.2 ms timepoint (start of the push corresponds to zero time point). HIFU is incident from the left of the images. Hashed dots on the vertical and solid dots on the horizontal axes denote the locations of SWE waveform detection.

FIG. 6D depicts waveforms of displacement for SW (low values) and LSW (high values) at different points on transverse and longitudinal axes, respectively.

FIG. 6E depicts normalized distribution of displacement or velocity along lateral coordinates over SW tracking time, known as “slow time”.

FIG. 6F depicts normalized distribution of displacement or velocity along axial coordinates over SW tracking time, known as “slow time”.

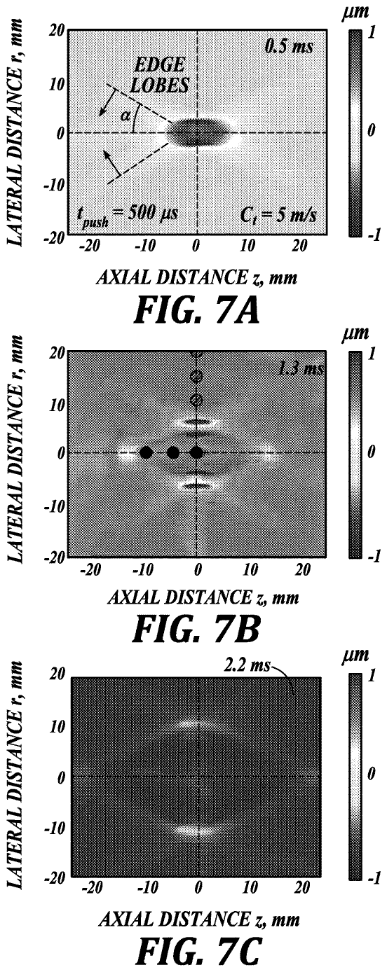

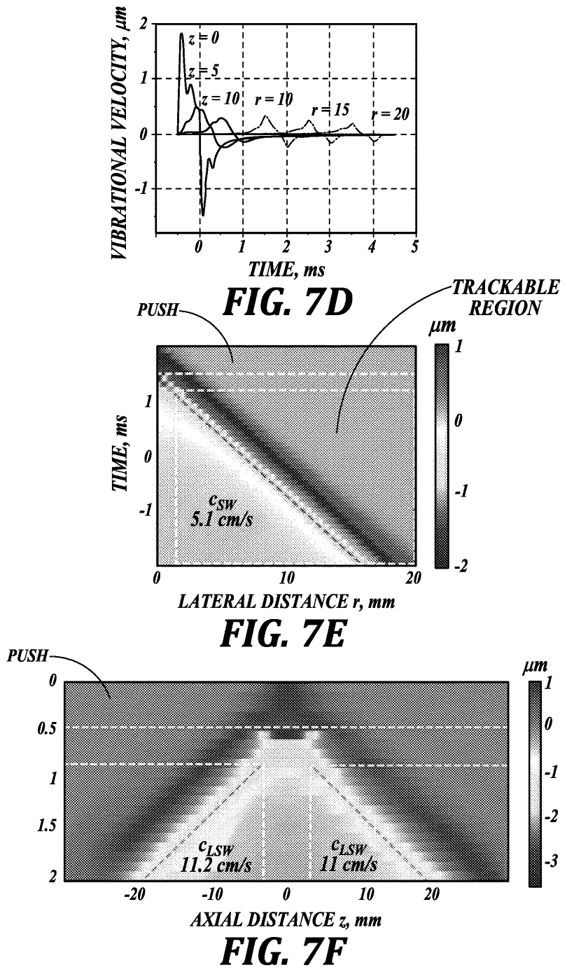

FIG. 7A depicts numerically simulated vibrational velocity fields in the phantom resulting from a 500 μs push from the HIFU transducer at a 0.5 ms timepoint (start of the push corresponds to zero time point). HIFU is incident from the left of the images. Hashed dots on the vertical and solid dots on the horizontal axes denote the locations of SWE waveform detection.

FIG. 7B depicts numerically simulated vibrational velocity fields in the phantom resulting from a 500 μs push from the HIFU transducer at a 1.3 ms timepoint (start of the push corresponds to zero time point). HIFU is incident from the left of the images. Hashed dots on the vertical and solid dots on the horizontal axes denote the locations of SWE waveform detection.

FIG. 7C depicts numerically simulated vibrational velocity fields in the phantom resulting from a 500 μs push from the HIFU transducer at a 2.2 ms timepoint (start of the push corresponds to zero time point). HIFU is incident from the left of the images. Hashed dots on the vertical and solid dots on the horizontal axes denote the locations of SWE waveform detection.

FIG. 7D depicts waveforms of vibrational velocity for SW (low values) and LSW (high values) at different points on transverse and longitudinal axes, respectively.

FIG. 7E depicts normalized distribution of displacement or velocity along lateral coordinates over SW tracking time, known as “slow time”.

FIG. 7F depicts normalized distribution of displacement or velocity along axial coordinates over SW tracking time, known as “slow time”.

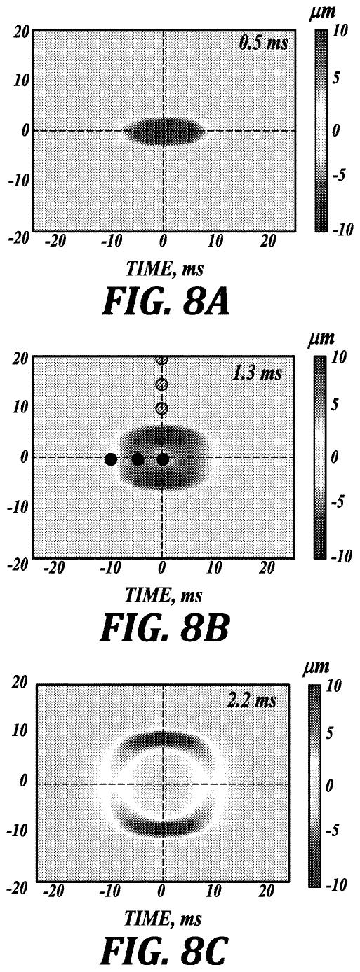

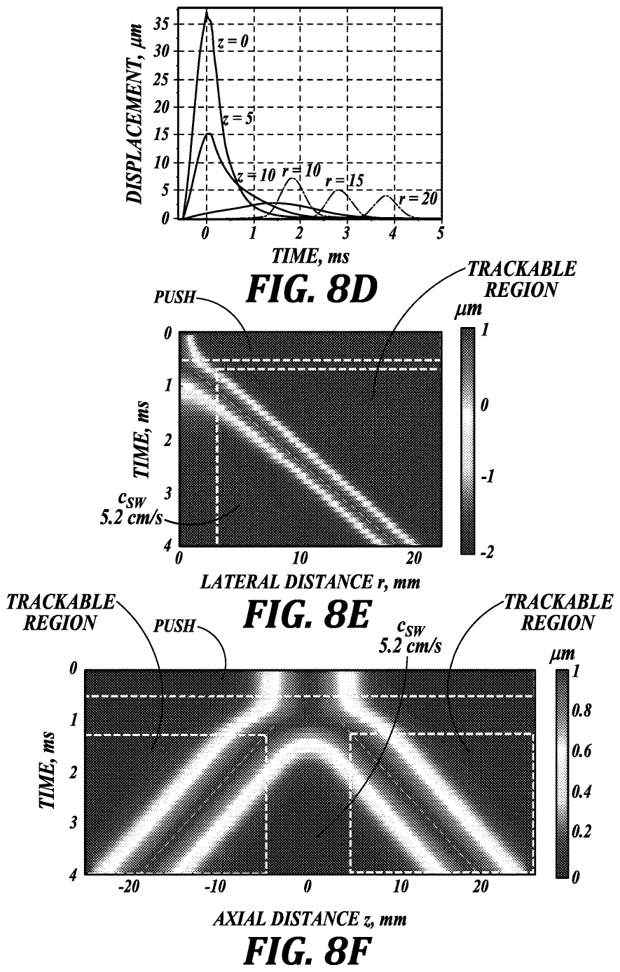

FIG. 8A depicts a Gaussian-shaped beam at a 0.5 ms timepoint (start of the push corresponds to zero time point). HIFU is incident from the left of the images. Hashed dots on the vertical and solid dots on the horizontal axes denote the locations of SWE waveform detection.

FIG. 8B depicts a Gaussian-shaped beam at a 1.3 ms timepoint (start of the push corresponds to zero time point). HIFU is incident from the left of the images. Hashed dots on the vertical and solid dots on the horizontal axes denote the locations of SWE waveform detection.

FIG. 8C depicts a Gaussian-shaped beam at a 2.2 ms timepoint (start of the push corresponds to zero time point). HIFU is incident from the left of the images. Hashed dots on the vertical and solid dots on the horizontal axes denote the locations of SWE waveform detection.

FIG. 8D depicts waveforms of displacement for SW (low values) and LSW (high values) at different points on transverse and longitudinal axes, respectively.

FIG. 8E depicts normalized distribution of displacement or velocity along lateral coordinates over SW tracking time, known as “slow time”.

FIG. 8F depicts normalized distribution of displacement or velocity along axial coordinates over SW tracking time, known as “slow time”.

The results of numerical modelling of SW generation and propagation are summarized in FIGS. 6A-FIG. 8F. As seen in FIG. 6A-FIG. 6F, the push generated with the HIFU transducer field corresponds to the intensity distribution in FIG. 5B and consists of a central elliptical region and lower amplitude cone-shaped edge lobes. Following the push, a SW starts propagating perpendicularly to the HIFU axis from the central push region with the prescribed shear wave speed of 5 m/s, as expected. The peak displacement following the push is only 5 μm, which is fairly low compared to displacements used in clinical SWE and ARFI imaging. This is due to a much lower absorption in tissue at the HIFU frequency of 1.5 MHz compared to >3 MHz typically used in US imaging probes. Interestingly, a SW of similar amplitude propagating along the HIFU axis at a speed almost twice higher is also observed. This SW will be referred to as longitudinal SW (LSW) hereinafter. LSW appears to be the result of constructive interference of shear waves excited prefocally and postfocally by the cone-shaped edge of the beam and propagating at the angle α=28° to the HIFU axis (FIG. 6A-FIG. 6F). The LSW speed is therefore expected to be c_{LSW}=c_{SW}/sin α=c_{SW}/0.47, which closely corresponds to the observations. This hypothesis is confirmed by the simulation of a focal Gaussian push region of similar dimensions, but lacking sharp beam edge—a situation more typical for pushes generated with imaging probes with aperture apodization (FIG. 8A-FIG. 8F). An LSW is also present in this case, but its amplitude decays with propagation distance very quickly, and it propagates at the same speed as the SW.

A well-known practical aspect of tracking SW propagation with time-of-flight (TOF) approach is that the SW velocity can only be measured correctly outside of the focal region of the push—approximately 2 mm on either side of the focus in present case. Likewise, the trackable region for LSW, i.e. the region within which a well-defined LSW is formed and propagates at a constant velocity, is outside of the axial extent of the focal lobe −5 mm to either side of the focus. Another practical aspect in SWE-ARFI is the choice of TOF method, with the two common ones being the measurement of the arrival time of displacement and vibrational velocity maxima. The latter case is simulated in FIG. 7A-FIG. 7F, and measurement of the arrival of the maximum negative velocity yields the same result for c_{SW} and c_{LSW} as does measurement of the arrival time of the maximum displacement in FIG. 6A-FIG. 6F. However, the absolute value of vibrational velocity (<2 cm/s) is likely to be very challenging to measure experimentally due to its increased susceptibility to noise.

### Example 3

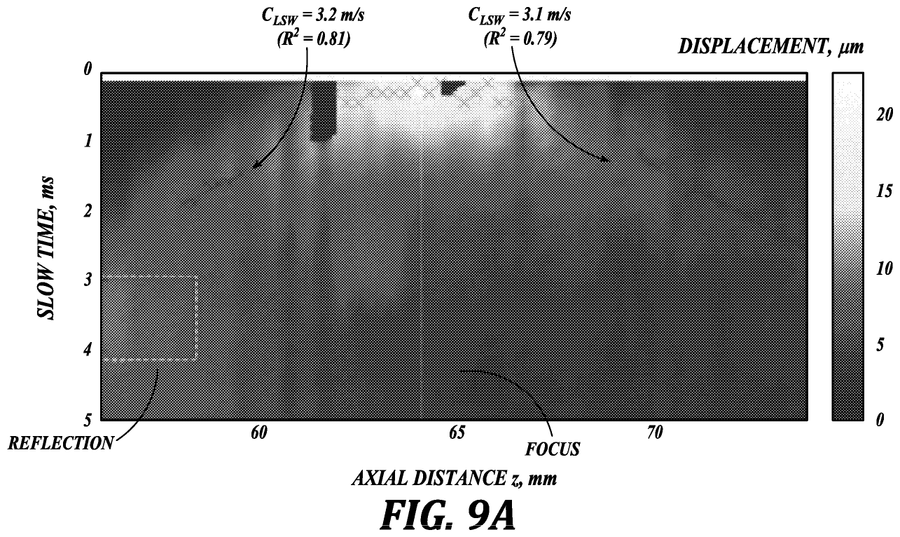

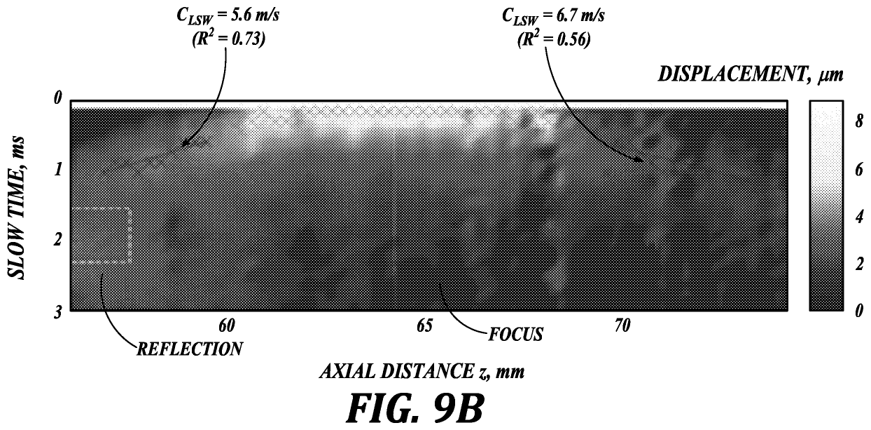

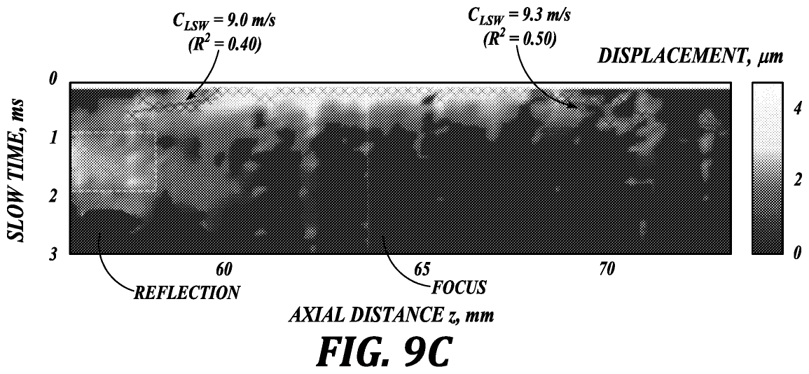

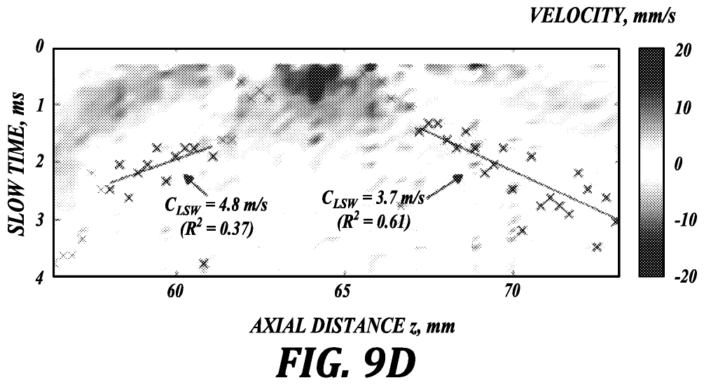

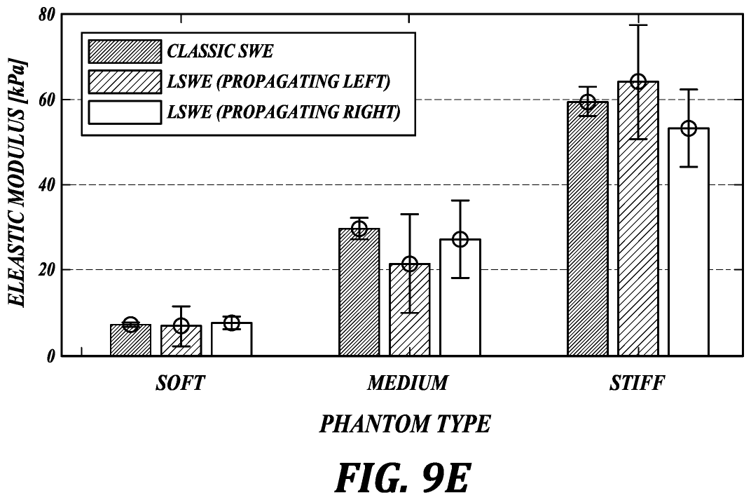

FIG. 9A depicts a measurement of the longitudinal shear wave speed c_{LSW} in homogenous phantoms of different stiffness. The arrival time of the maximum displacement (marked as black crosses) was measured at each location along the HIFU axis and c_{LSW} was obtained through linear regression (black lines) in soft phantoms. R² is the coefficient of determination for each regression.

FIG. 9B depicts a measurement of the longitudinal shear wave speed c_{LSW} in homogenous phantoms of different stiffness. The arrival time of the maximum displacement (marked as black crosses) was measured at each location along the HIFU axis and c_{LSW} was obtained through linear regression (black lines) in medium phantoms. R² is the coefficient of determination for each regression.

FIG. 9C depicts a measurement of the longitudinal shear wave speed c_{LSW} in homogenous phantoms of different stiffness. The arrival time of the maximum displacement (marked as black crosses) was measured at each location along the HIFU axis and c_{LSW} was obtained through linear regression (black lines) in stiff phantoms. R² is the coefficient of determination for each regression.

FIG. 9D depicts the arrival time of maximum negative vibrational velocity used to obtain c_{LSW} in the soft phantom due to very low signal-to-noise ratio (SNR). Grey crosses in all cases correspond to the locations that were either outside of the trackable area or did not meet the cutoff SNR criteria and were thus not included in the linear regression.

FIG. 9E depicts an average Young's moduli of the three phantoms measured by the clinical SWE system (narrow hatching, “classic SWE”) and calculated from c_{LSW} of the waves propagating towards (wide hatching) and away (white) from the transducer as E=3ρ(0.47c_{LSW})² are in a good agreement. The error bars indicate standard deviation for n=5 measurements.

FIG. 9A-FIG. 9C illustrate the process of c_{LSW} measurement in the three phantoms, from the softest to the stiffest. The arrival time of the maximum displacement at each location on the axis was measured within the trackable region, i.e. outside of the focal area, and the c_{LSW} was calculated via linear regression across those points. Thus, two measurements, for LSWs propagating away from the transducer and towards it were obtained. Shear wave velocity was then calculated as c_{SW}=0.47c_{LSW}, and Young's modulus as

$$E = 3 ρ {c}_{SW}^{2}$$

to compare to the clinical system measurements. A reflection of the LSW from the phantom-water interface was also observed for the LSWs propagating towards the transducer, and the corresponding locations were not included in the linear regression. As expected, the absolute values for displacement were highest for the softest phantom, and lowest for the stiffest one, ranging within 4-22 microns. Accordingly, the measurement SNR and R² in regression fits progressively deteriorated with increasing phantom stiffness.

A measurement of c_{LSW} from tracking the propagation of the maximum of vibrational velocity (specifically, the negative vibrational velocity) was also attempted in the softest phantom and is illustrated in FIG. 9D. Because vibrational velocity estimation requires numerical differentiation of displacement—amplifying high-frequency jitter and reducing effective SNR—the resulting fits in determining c_{LSW} exhibited greater uncertainty (lower R²) than displacement-based fits. This issue was even more exacerbated in stiffer phantoms (data not shown). Thus, the arrival time of the maximum of displacement was used thereafter. The summary of the phantoms' Young's moduli calculated from c_{LSW} measurements in different locations (n=5) of the phantoms and from the clinical SWE imaging is presented in FIG. 9E. The LSW-derived elasticity values are generally in agreement with those measured with clinical SWE imaging, although the measurement errors are larger in both techniques for stiffer phantoms, which is expected due to smaller displacements and, consequently, lower SNR. Notably, the prostate tissue absorption at 1.5 MHz (~0.14 NP/cm) is higher than in the phantom used in this study (~0.05 NP/cm). Because acoustic radiation force scales with absorption, the same focal intensity would produce larger displacements and higher effective SNR in tissue, thereby reducing the uncertainty observed for the stiffer phantom.

### Example 4

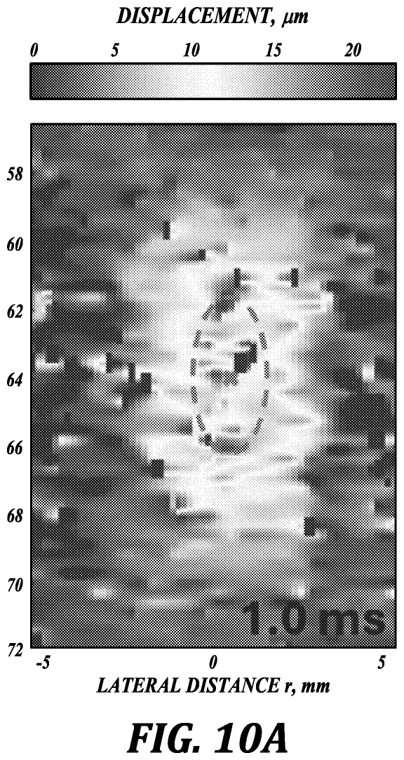

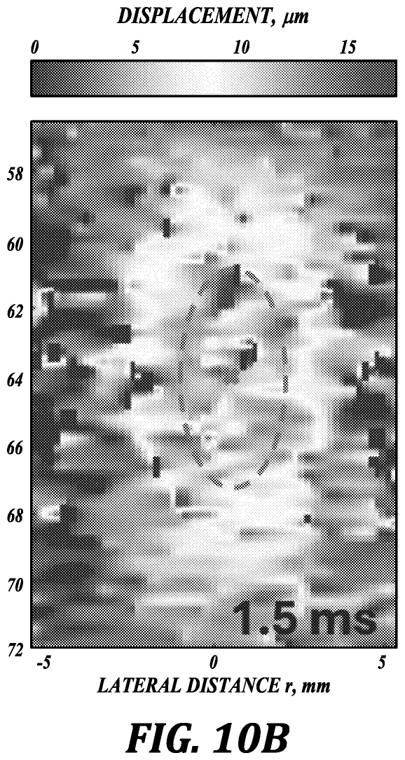

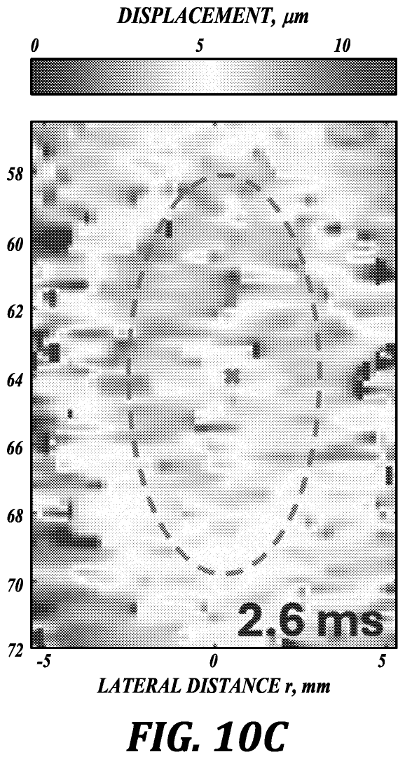

FIG. 10A-FIG. 10C depicts direct plane wave imaging of shear wave propagation following a HIFU push in the softest phantom. The distribution of displacement is captured at 1 ms (FIG. 10A), 1.5 ms (FIG. 10B), and 2.6 ms (FIG. 10C) after the end of the push. The gray cross corresponds to the HIFU focus. The ratio of the axes of the dashed gray oval corresponds to the ratio of SW and LSW velocities: c_{SW}/c_{LSW}=0.47 predicted by numerical modelling.

The prediction that LSW velocity is approximately twice higher than that of SW was additionally confirmed by directly observing the propagation of shear waves following a HIFU push in the softest phantom. FIG. 10A-10C shows the distribution of displacement at three time points (1.0 ms, 1.5 ms and 2.6 ms) following the end of the push. As seen, the SW wavefront is oval-shaped, with the ratio of the axes corresponding closely to c_{SW}/c_{LSW}=0.47 predicted by numerical modeling.

The HIFU-LSWEI technique of mapping tissue elasticity demonstrated here has a number of practical advantages.

First, it can be realized with the most widely used US-guided HIFU arrangement: a single-element focused transducer (or an annular array) coaxially integrated with a limited aperture imaging probe that uses a single central ray line for the measurement. In fact, even a single-element pulse-echo probe could be used in place of the imaging array in applications where target visualization is not required, further simplifying the system.

Second, the SNR of displacement measurements is higher than that achievable in the same arrangement using 2D tracking (FIG. 10A-10C). In our measurements, the mean absolute value of displacement noise obtained by cross-correlating two pre-push reference pulses was 0.22±0.21 μm for 1D focused tracking and 0.53±0.65 μm for 2D planewave tracking with low bias in both cases but 2.7 times higher jitter for 2D (0.31 vs 0.84 μm). Given these noise characteristics, SNR for the 1D tracking was 33.8 dB, whereas SNR for the 2D tracking was 30.0 dB.

Third, the limited angular aperture of the imaging probe within the region of interest substantially reduces the lateral spatial resolution, complicating the 2D tracking of SW, but not the axial resolution and 1D tracking of LSW.

Lastly, the measurement is time-efficient, as it provides the distribution of tissue stiffness along the HIFU axis within a large range with only a single push.

In this regard, in some embodiments, mapping a tissue stiffness of the biological tissue includes comparing tissue stiffness at a plurality of zones in the tissue of the patient. For instance, the HIFU system may be positioned at a first external position of a patient. Thereafter, a tissue stiffness may be determined at a first zone, a second zone, a third zone, etc., by repositioning the push region within the patient in any of the manners described herein (e.g., by focusing the phased array ultrasound transducer to a push region of the first zone, to the push region of the second zone, etc.). By determining tissue stiffness at a first zone, a second, zone, a third zone, and the like, tissue stiffness in the tissue of the patient may be mapped, thereby mapping the presence of abnormalities in the biological tissue within a 2D plane. Furthermore, the HIFU system may thereafter be positioned to a second external position of the patient, and a plurality of zones in the tissue of the patient mapped from this second external position as described for the first external position, thereby generating a second mapping of the presence of abnormalities in the biological tissue within a second 2D plane. By repeating this process, a 3D map of abnormalities in the biological tissue may be generated. Thus, in some embodiments, “mapping a tissue stiffness” may include 2D mapping of tissue from a single external position of a tissue, 2D mapping of tissue from multiple external positions of a tissue, 3D mapping of tissue, and/or combinations thereof.

In conclusion, the current study has demonstrated, both numerically and experimentally, the generation of LSWs by a HIFU push that propagate at much higher velocity than SW propagating laterally. The origin of those “supersonic” LSW appears to be constructive interference of shear waves excited by the cone-shaped edge of the HIFU beam. Tracking the propagation of LSWE by a coaxial US imaging probe allows for a rapid measurement of tissue elasticity distribution along the HIFU axis. This technique could be useful in mapping the boundaries of stiff inclusions in tissue for HIFU intervention planning.

The detailed description set forth above in connection with the appended drawings, where like numerals reference like elements, are intended as a description of various embodiments of the present disclosure and are not intended to represent the only embodiments. Each embodiment described in this disclosure is provided as a representative example or illustration and should not be construed as preferred or advantageous over other embodiments. The representative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification. That is, the present disclosure includes embodiments that combine features from different embodiments.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

In the detailed description herein, references to “one embodiment”, “some embodiments”, “an embodiment”, “an example embodiment”, “one or more embodiments, etc., indicate that the embodiment described may include a particular feature, structure, or characteristic, but every embodiment may not necessarily include the particular feature, structure, or characteristic. Moreover, such phrases are not necessarily referring to the same embodiment. In addition, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments. Thus, it will be appreciated that embodiments of the present disclosure may employ any combination of features described herein. All such combinations or sub-combinations of features are within the scope of the present disclosure.

In view of the limitations of the processing techniques available in the field, the terms “approximately” and “about” reflect a certain inability (or uncertainty) to precisely control the exact dimensions of certain features and measurements described herein. Depending on the level of precision that can be achieved using the commercially available processing and measurement tools available at the time, the terms “approximately” and “about” may be used to mean within ±5% of a target value. The terms “approximately” and “about” may include the target value.

Throughout this specification, terms of art may be used. These terms are to take on their ordinary meaning in the art from which they come, unless specifically defined herein or the context of their use would clearly suggest otherwise.

The drawings in the FIGURES are not to scale. Similar elements are generally denoted by similar references in the FIGURES. For the purposes of this disclosure, the same or similar elements may bear the same references. Furthermore, the presence of reference numbers or letters in the drawings cannot be considered limiting, even when such numbers or letters are indicated in the claims.

Terms such as “a,” “an,” “the,” and “said” are used to indicate the presence of one or more elements and components. The terms “comprise,” “include,” “have,” “contain,” and their variants are used to be open ended and may include or encompass additional elements, components, etc., in addition to the listed elements, components, etc., unless otherwise specified. The terms “first,” “second,” etc. may be used as differentiating identifiers of individual or respective components among a group thereof, rather than as a descriptor of a number of the components, unless clearly indicated otherwise.

Although relative terms such as “on,” “below,” “upper,” “lower,” “top,” “bottom,” “right,” and “left” may be used to describe the relative spatial relationships of certain structural features, these terms are used for convenience only, as a direction in the examples. Thus, if a structure is turned upside down, the “upper” component will become a “lower” component. When a structure or feature is described as being “on” (or formed on) another structure or feature, the structure can be positioned directly on (i.e., contacting) the other structure, without any other structures or features intervening between the structure and the other structure. When a structure or feature is described as being “over” (or formed over) another structure or feature, the structure can be positioned over the other structure, with or without other structures or features intervening between them.

When two components are described as being “coupled to” each other, the components can be electrically coupled to each other, with or without other components being electrically coupled and intervening between them. When two components are described as being “directly coupled to” each other, the components can be electrically coupled to each other, without other components being electrically coupled between them.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting embodiments contain elements that are modular and can be combined with each other in any number, order, or combination to form a new non-limiting embodiment, which can itself be further combined with other non-limiting embodiments.

Embodiment 1. A method for classifying biological tissue using high intensity focused ultrasound (HIFU), the method including: displacing the biological tissue by a burst of ultrasound transmitted by a first ultrasound transducer, wherein the first ultrasound transducer is a high intensity focused ultrasound (HIFU) transducer; by displacing the biological tissue, generating a shear wave propagation pattern in the biological tissue, wherein the shear wave propagation pattern includes a longitudinal shear wave pattern and a normal shear wave pattern; tracking the shear wave propagation pattern by a second ultrasound transducer that is an imaging ultrasound transducer; and by tracking the shear wave propagation pattern, mapping a tissue stiffness of the biological tissue.

Embodiment 2. The method of Embodiment 1 or any other Embodiment, wherein operational parameters of the first ultrasound transducer include a duration of the burst of between about 50 μs and about 500 μs.

Embodiment 3. The method of any of Embodiments 1-2 or any other Embodiment, wherein operational parameters of the first ultrasound transducer include a frequency of the first ultrasound transducer that is lower than a frequency of the imaging transducer.

Embodiment 4. The method of any of Embodiments 1-3 or any other Embodiment, wherein the second ultrasound transducer is configured for collimated A-line ultrasound imaging.

Embodiment 5. The method of any of Embodiments 1-4 or any other Embodiment, wherein a tissue abnormality is present when a speed of the longitudinal shear wave pattern is above a predetermined threshold.

Embodiment 6. The method of any of Embodiments 1-5 or any other Embodiment, wherein the tissue abnormality is indicative of a fibrous tissue characterized by a first stiffness that is higher than a second stiffness of surrounding tissue.

Embodiment 7. The method of any of Embodiments 1-6 or any other Embodiment, wherein the tissue abnormality is indicative of a tumor tissue characterized by a first stiffness that is higher than a second stiffness of surrounding tissue.

Embodiment 8. The method of any of Embodiments 1-7 or any other Embodiment, wherein the tumor is a prostate tumor.

Embodiment 9. The method of any of Embodiments 1-8 or any other Embodiment, wherein tracking the shear wave propagation pattern consists of tracking the longitudinal shear wave pattern.

Embodiment 10. The method of any of Embodiments 1-9 or any other Embodiment, wherein a tissue abnormality is present when signal attenuation of the longitudinal shear wave pattern is above a predetermined threshold.

Embodiment 11. The method of any of Embodiments 1-10 or any other Embodiment, wherein the tissue abnormality is indicative of a tissue having a first viscosity that is higher than a second viscosity of a surrounding tissue.

Embodiment 12. The method of any of Embodiments 1-11 or any other Embodiment, further including identifying a location of a tissue abnormality based on an elastic modulus of the biological tissue.

Embodiment 13. The method of any of Embodiments 1-12 or any other Embodiment, wherein the location is obtained from a plurality of time-offset A-line images.

Embodiment 14. The method of any of Embodiments 1-13 or any other Embodiment, further including identifying a type of the tissue abnormality based on the elastic modulus of the biological tissue.

Embodiment 15. The method of any of Embodiments 1-14 or any other Embodiment, wherein the second ultrasound transducer is configured for tracking the shear wave propagation pattern based on a speckle displacement.

Embodiment 16. The method of any of Embodiments 1-15 or any other Embodiment, wherein the speckle displacement is indicative of tissue movement.

Embodiment 17. The method of any of Embodiments 1-16 or any other Embodiment, wherein the speckle displacement takes place over a time window of about 1 ms to about 50 ms, or of about 1 ms to about 10 ms.

Embodiment 18. The method of any of Embodiments 1-17 or any other Embodiment, wherein the HIFU transducer is a phased array HIFU transducer.

Embodiment 19. The method of any of Embodiments 1-18 or any other Embodiment, wherein the biological tissue is displaced away from a center axis of the phased array HIFU transducer.

Embodiment 20. The method of any of Embodiments 1-19 or any other Embodiment, wherein the imaging transducer is focused away from the center axis of the phased array HIFU transducer.

Embodiment 21. The method of any of Embodiments 1-20 or any other Embodiment, wherein the imaging transducer is a single element transducer.

Embodiment 22. The method of any of Embodiments 1-21 or any other Embodiment, wherein the imaging transducer is a phased array transducer.

Embodiment 23. The method of any of Embodiments 1-22 or any other Embodiment, wherein displacing the biological tissue by a burst of ultrasound transmitted takes place over a first range of scanning angles.

Embodiment 24. The method of any of Embodiments 1-23 or any other Embodiment, wherein the first range of scanning angles is between about 5° with respect to the acoustic axis and about 45° with respect to the acoustic axis.

Embodiment 25. The method of any of Embodiments 1-24 or any other Embodiment, wherein the HIFU transducer is configured to generate a HIFU beam having a distribution of pressure that includes a plurality of edge lobes.

Embodiment 26. The method of any of Embodiments 1-25 or any other Embodiment, wherein the plurality of edge lobes are axially symmetric in at least one dimension.

Embodiment 27. A method for classifying biological tissue using high intensity focused ultrasound (HIFU), the method including: based on a prior imaging, placing an ultrasound probe on patient's body proximate to the biological tissue of interest, the ultrasound probe including a first ultrasound transducer and a second ultrasound transducer; displacing the biological tissue by a plurality of bursts of ultrasound transmitted by the first ultrasound transducer, wherein the first ultrasound transducer is a high intensity focused (HIFU) transducer, wherein individual bursts of the plurality of bursts are characterized by differing angles or focal areas; by displacing the biological tissue by individual bursts of ultrasound, generating corresponding shear wave propagation pattern in the biological tissue, wherein the shear wave propagation pattern includes longitudinal shear wave pattern and a normal shear wave pattern; tracking the shear wave propagation pattern by the second ultrasound transducer that is an imaging ultrasound transducer; and by tracking the shear wave propagation pattern, mapping a tissue stiffness of the biological tissue.

Embodiment 28. The method of Embodiment 27 or any other Embodiment, wherein the biological tissue is a prostate tissue of a patient.

Embodiment 29. The method of any of Embodiments 27-28 or any other Embodiment, wherein mapping the tissue stiffness maps: a stiffness value and a location of an area with increased stiffness.

Embodiment 30. The method of any of Embodiments 27-29 or any other Embodiment, wherein the prior imaging is a CTE imaging or an MRI imaging.

Embodiment 31. A system for classifying biological tissue using high intensity focused ultrasound (HIFU), the system including: a first ultrasound transducer that is a high intensity focused (HIFU) transducer configured for: displacing the biological tissue by transmitting a burst of ultrasound, and by displacing the biological tissue, generating a shear wave propagation pattern in the biological tissue, wherein the shear wave propagation pattern includes longitudinal shear wave pattern and a normal shear wave pattern; and a second ultrasound transducer that is an imaging ultrasound transducer, wherein the second ultrasound transducer is configured for mapping an elastic modulus of the biological tissue by tracking the shear wave propagation.

Embodiment 32. The system of Embodiment 31 or any other Embodiment, further including: a controller configured for identifying a location of abnormal tissue based on the elastic modulus of the biological tissue.

Embodiment 33. The system of any of Embodiments 31-32 or any other Embodiment, wherein the controller is further configured for: identifying a size and a shape of the abnormal tissue based on the elastic modulus of the biological tissue.

Embodiment 34. The system of any of Embodiments 31-33 or any other Embodiment, wherein the controller is further configured for: identifying a type of abnormal tissue based on the elastic modulus of the biological tissue.

Embodiment 35. The system of any of Embodiments 31-34 or any other Embodiment, wherein the second ultrasound transducer is configured for tracking the shear wave propagation pattern based on speckle displacement.

Embodiment 36. The system of any of Embodiments 31-35 or any other Embodiment, wherein the speckle displacement takes place over a time window of about 1 ms to about 50 ms, or about 1 ms to about 10 ms.

Embodiment 37. The system of any of Embodiments 31-36 or any other Embodiment, wherein the HIFU transducer is a phased array HIFU transducer.

Embodiment 38. The system of any of Embodiments 31-37 or any other Embodiment, wherein the phased array HIFU transducer is configured for displacing the biological tissue away from a center axis of the phased array HIFU transducer.

Embodiment 39. The system of any of Embodiments 31-38 or any other Embodiment, wherein the imaging ultrasound transducer is focused away from the center axis of the phased array HIFU transducer.

Embodiment 40. The system of any of Embodiments 31-39 or any other Embodiment, wherein the imaging ultrasound transducer is a single element ultrasound transducer.

Embodiment 41. The system of any of Embodiments 31-40 or any other Embodiment, wherein the HIFU transducer is configured to generate a HIFU beam having a distribution of pressure that includes a plurality of edge lobes.

Embodiment 42. The system of any of Embodiments 31-41 or any other Embodiment, wherein the plurality of edge lobes are axially symmetric in at least one dimension.

While illustrative embodiments have been illustrated and described, it will be appreciated that various changes can be made therein without departing from the spirit and scope of the invention.

What is claimed is:

1. A method for classifying biological tissue using high intensity focused ultrasound (HIFU), the method comprising:
displacing the biological tissue by a burst of ultrasound transmitted by a first ultrasound transducer, wherein the first ultrasound transducer is a high intensity focused ultrasound (HIFU) transducer;
by displacing the biological tissue, generating a shear wave propagation pattern in the biological tissue, wherein the shear wave propagation pattern comprises a longitudinal shear wave pattern and a normal shear wave pattern;
tracking the shear wave propagation pattern by a second ultrasound transducer that is an imaging ultrasound transducer; and
by tracking the shear wave propagation pattern, mapping a tissue stiffness of the biological tissue.

2. The method of claim 1, wherein operational parameters of the first ultrasound transducer comprise a duration of the burst of between about 50 μs and about 500 μs.

3. The method of claim 1, wherein operational parameters of the first ultrasound transducer comprise a frequency of the first ultrasound transducer that is lower than a frequency of the imaging transducer.

4. The method of claim 1, wherein the second ultrasound transducer is configured for collimated A-line ultrasound imaging.

5. The method of claim 1, wherein a tissue abnormality is present when a speed of the longitudinal shear wave pattern is above a predetermined threshold.

6. The method of claim 1, wherein tracking the shear wave propagation pattern consists of tracking the longitudinal shear wave pattern.

7. The method of claim 1, wherein a tissue abnormality is present when signal attenuation of the longitudinal shear wave pattern is above a predetermined threshold.

8. The method of claim 1, further comprising identifying a location of a tissue abnormality based on an elastic modulus of the biological tissue.

9. The method of claim 1, wherein the HIFU transducer is a phased array HIFU transducer.

10. The method of claim 1, wherein the imaging transducer is a single element transducer.

11. The method of claim 1, wherein the imaging transducer is a phased array transducer.

12. The method of claim 1, wherein the HIFU transducer is configured to generate a HIFU beam having a distribution of pressure that comprises a plurality of edge lobes.

13. A method for classifying biological tissue using high intensity focused ultrasound (HIFU), the method comprising:
based on a prior imaging, placing an ultrasound probe on patient's body proximate to the biological tissue of interest, the ultrasound probe comprising a first ultrasound transducer and a second ultrasound transducer;
displacing the biological tissue by a plurality of bursts of ultrasound transmitted by the first ultrasound transducer, wherein the first ultrasound transducer is a high intensity focused (HIFU) transducer, wherein individual bursts of the plurality of bursts are characterized by differing angles or focal areas;
by displacing the biological tissue by individual bursts of ultrasound, generating corresponding shear wave propagation pattern in the biological tissue, wherein the shear wave propagation pattern comprises longitudinal shear wave pattern and a normal shear wave pattern;
tracking the shear wave propagation pattern by the second ultrasound transducer that is an imaging ultrasound transducer; and
by tracking the shear wave propagation pattern, mapping a tissue stiffness of the biological tissue.

14. A system for classifying biological tissue using high intensity focused ultrasound (HIFU), the system comprising:
a first ultrasound transducer that is a high intensity focused (HIFU) transducer configured for:
displacing the biological tissue by transmitting a burst of ultrasound, and
by displacing the biological tissue, generating a shear wave propagation pattern in the biological tissue, wherein the shear wave propagation pattern comprises longitudinal shear wave pattern and a normal shear wave pattern; and
a second ultrasound transducer that is an imaging ultrasound transducer, wherein the second ultrasound transducer is configured for mapping an elastic modulus of the biological tissue by tracking the shear wave propagation.

15. The system of claim 14, further comprising:
a controller configured for identifying a location of abnormal tissue based on the elastic modulus of the biological tissue.

16. The system of claim 15, wherein the controller is further configured for:
identifying a size and a shape of the abnormal tissue based on the elastic modulus of the biological tissue.

17. The system of claim 15, wherein the controller is further configured for:
identifying a type of abnormal tissue based on the elastic modulus of the biological tissue.

18. The system of claim 14, wherein the HIFU transducer is a phased array HIFU transducer.

19. The system of claim 14, wherein the imaging ultrasound transducer is a single element ultrasound transducer.

20. The system of claim 14, wherein the HIFU transducer is configured to generate a HIFU beam having a distribution of pressure that comprises a plurality of edge lobes.
